(19) United States
(12) Patent Application Publication
(10) Pub. No.: US 2021/0330010 A1
(43) Pub. Date: Oct. 28, 2021

(54) DISPOSABLE ANTIVIRAL AND ANTIBACTERIAL GLOVES

(71) Applicant: Eileen Waldron, Charleston, SC (US)

(72) Inventor: Eileen Waldron, Charleston, SC (US)

(21) Appl. No.: 17/301,817

(22) Filed: Apr. 15, 2021

Related U.S. Application Data
(60) Provisional application No. 63/016,458, filed on Apr. 28, 2020.

(51) Int. Cl. A41D 31/30; A41D 19/015; A41D 19/01; A61K 8/02; A61K 8/73; A61K 8/34; A61K 8/81; A61K 8/86; A61K 8/22; A61K 8/33; A61Q 17/00; C08L 67/00; C08L 23/12; C08L 97/02; C08L 1/02

(52) U.S. Cl. CPC A41D 31/30; A61B 42/10; A41D 19/01; A61K 8/0208; A61K 8/731; A61K 8/34; A61K 8/8111; A61K 8/86; A61K 8/22; A61K 8/33; A61Q 17/005; C08L 67/00; C08L 23/12; C08L 97/02; C08L 1/02; A41D 19/015

(57) ABSTRACT

An antibacterial glove of the disclosure includes a woven or nonwoven absorbent or semi-absorbent substrate material infused with a disinfectant composition, wherein the glove includes one or more layers, and each layer comprises the woven or nonwoven absorbent or semi-absorbent substrate material. The antibacterial glove may include a hand sanitizing composition impregnated therein. The disinfectant composition may include an alcohol, hydrogen peroxide, and/or hypochlorite. The woven or nonwoven absorbent or semi-absorbent substrate material may include a cellulosic fiber and/or a thermoplastic fiber.

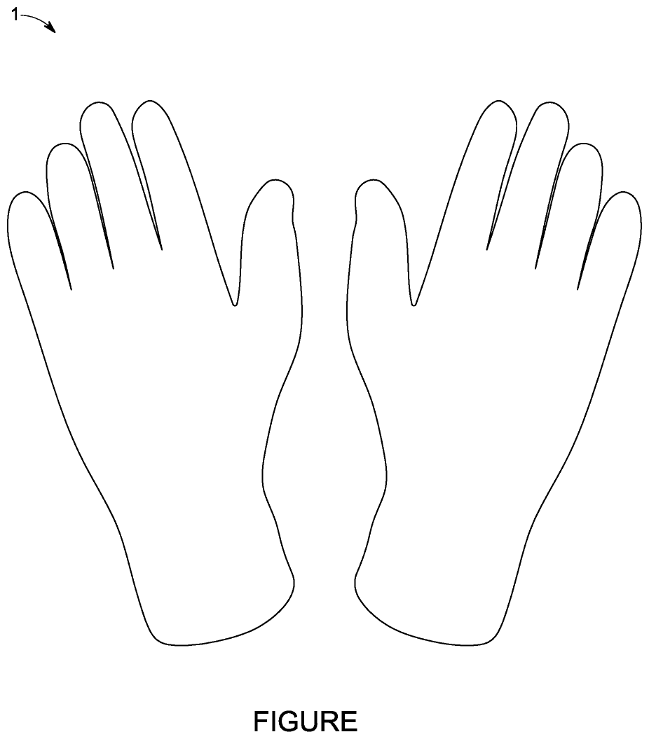

### CROSS REFERENCE TO RELATED APPLICATIONS

This application claims priority to U.S. provisional application No. 63/016,458, filed on Apr. 28, 2020, which is herein incorporated by reference in its entirety.

### BACKGROUND OF THE DISCLOSURE

### Field of the Invention

This disclosure is generally directed to the field of gloves including disposable gloves, more particularly, antibacterial and/or antiviral gloves that provide protection to a user's hands from bacteria or viruses on various surfaces including doorknobs, credit card payment devices, etc., in a user's home, or elsewhere in places of business, or other public places.

### Description of the Related Art

Infections caused by the transfer of nosocomial pathogens from high-touch environmental surfaces and medical devices are responsible for significant morbidity, mortality, and economic cost. It is known that pathogens, including Staphylococcus aureus (MRSA), norovirus, Clostridium difficile, vancomycin-resistant Enterococcus, Acinetobacter species etc., and the SARS2-COV2 virus can contaminate surfaces at concentrations adequate for transmission. Such bacteria can also survive for extended periods and persist despite attempts to remove them.

Disinfecting wipes including impregnated disinfecting wipes, pre-saturated towelette and pre-wetted disinfecting wipes are known for decontamination of high-touch surfaces because of their convenience and reliable performance. Disinfectant-impregnated wipes basically include an absorbent towel-type material saturated with diluted disinfectant as well as other chemical products such as surfactants, preservatives, enzymes, and perfumes etc.

People often use hand gloves when they go shopping or out into public. This has become particularly true during the covid-19 crisis. In fact, however, ordinary latex, vinyl, neoprene, rubber, acrylonitrile, polyvinyl chloride, etc., gloves do not provide sufficient protection from bacteria, viruses, etc. on ordinary surfaces. It is well-known that the COVID-19-19 virus can remain active on a surface for a significant period. Ordinary gloves therefore will not sufficiently protect an individual from bacteria, viruses, etc., that exist on surfaces in public places.

For this reason, in the recent Covid-19 crisis, for example, many people have been using antiviral-bacterial wipes when they go shopping or go out into public to clean surfaces before touching them. This, however, is cumbersome and difficult and it is generally not possible to adequately clean every surface before it is touched. Accordingly, better methods are needed to protect an individual's hands from bacteria, viruses, etc., on surfaces at home or in public places.

### SUMMARY OF THE INVENTION

An antibacterial glove of the disclosure includes a woven or nonwoven absorbent or semi-absorbent substrate material infused with a disinfectant composition, wherein the glove includes one or more layers, and each layer comprises the woven or nonwoven absorbent or semi-absorbent substrate material. The antibacterial glove may include a hand sanitizing composition impregnated therein. The disinfectant composition may include an alcohol, hydrogen peroxide, and/or hypochlorite. The woven or nonwoven absorbent or semi-absorbent substrate material may include a cellulosic fiber and/or a thermoplastic fiber.

The present disclosure is thus directed to hand gloves made from materials analogous to antibacterial/antiviral wipes. Analogous to antibacterial/antiviral wipes, the gloves are infused or impregnated with an antibacterial or antiviral composition. The gloves may be conveniently stored in a plastic box or container and pulled individually as needed for use. The inventive gloves may be disposable. An individual's hands would then be protected from whatever is touched on any surface without having to pre-wipe the surface.

Other features and aspects will be apparent from the following detailed description, the FIGURE, and the claims.

### BRIEF DESCRIPTION OF THE DRAWINGS

The FIGURE shows an embodiment of a pair of gloves of the disclosure.

Throughout the drawings and the detailed description, the same reference numerals refer to the same elements. The drawings may not be to scale, and the relative size, proportions, and depiction of elements in the drawings may be exaggerated for clarity, illustration, and convenience.

### DETAILED DESCRIPTION

The following detailed description is provided to assist the reader in gaining a comprehensive understanding of the methods, products, and/or systems, described herein. However, various changes, modifications, and equivalents of the methods, products, and/or systems described herein will be apparent to an ordinary skilled artisan.

The disclosure is directed to gloves including absorbent or semi absorbent synthetic fibers or textile materials, soaked in virus and bacteria killing compounds. The glove material could include cellulosic fibers (cotton, wood pulp, lyocell, viscose) and/or thermoplastic fibers (polypropylene, polyethylene terephthalate) or any materials that are used in hand wipes.

For example, a nonwoven, water-insoluble, substrate for the gloves may be made from plant-grown materials or may be manufactured from processed plant-grown materials or may be synthetic manmade materials. The nonwoven substrate can be made from a material such as viscose, rayon, polyester, wood pulp, polypropylene, polyethylene, nylon, and/or cotton.

In embodiments, the basis weight of the substrate material for the gloves may be from about 10 grams per square meter (gsm) to about 200 gsm, or from about 20 gsm to about 100 gsm or 50 to 90 gsm. A higher basis weight range would allow higher loading capacity and heavier duty capabilities, for example, for applications in a hospital setting.

In embodiments, the substrate for the gloves may be loaded with the disinfecting composition at the loading level from about 1.0 times the dry weight of the wipe to about 10 times the dry weight of the glove, or from about 2.5 to about 7.5, or from about 3 to about 6 times the dry weight of the glove.

The gloves may come in a variety of shapes, including but not limited to, regular gloves, tight fitting or loose fitting, mittens, or even bags, etc., that can fit over and protect the hand. The gloves may cover the wrist and have, for example, wrist ties, Velcro straps, or some other like convenient means to secure the gloves particularly around the wrist and protect the hand.

Each individual glove may be arranged in a folded configuration and stacked one on top of the other to provide a stack of gloves. Alternatively, the gloves can be configured as a stack or roll for dispensing.

The virus and bacteria killing compounds of a disinfectant could include ethyl benzyl dimethyl ammonium, dimethyl benzyl ammonium chloride, ethyl benzyl ammonium chloride, quaternary ammonium, or quaternary ammonium salts or any compounds deemed to be effective based on EPA guidelines. See Disinfectant, Wikipedia, The Free Encyclopedia, Date of last revision: 6 Apr. 2021, herein incorporated by reference.

Quaternary ammonium compounds useful for preparing antimicrobial gloves of the disclosure include octyl decyl dimethyl ammonium chloride, dioctyl dimethyl ammonium chloride, didecyl dimethyl ammonium chloride, dimethyl ethyl benzyl ammonium chloride, didecyl dimethyl ammonium carbonate, didecyl dimethyl ammonium bicarbonate, benzethonium chloride, and combinations thereof. Antimicrobial gloves of the invention may further be treated with dye, fragrance, alcohol, or any combination thereof.

Alcohol based compositions may also be used. Compositions containing alcohols such as ethanol, isopropanol or n-propanol are contemplated. Preferred alcohol compositions contain 60 to 95 percent alcohol usually with water as the major secondary ingredient.

The gloves, impregnated with a disinfectant antiviral or microbial composition, may be provided in a plastic container with a resealable open and closed flap. The gloves fit either hand and could come in a variety of different sizes. The gloves may be for consumer use, e.g., for shopping in stores, handling shopping carts, choosing products, and checking out on a keypad. They could also be used for employees to stock shelves. They could also be used in a hospital or medical environment. After use they may be disposed.

As mentioned, the material for the gloves may include textile materials, including, but not limited to, cellulosic fibers (e.g., cotton, wood pulp, viscose, lyocell) and thermoplastic fibers (e.g., polyethylene terephthalate, and polypropylene). Particularly, the raw materials may include cellulosic fibers and polyolefin fibers. Cellulosic fibers provide high water retention and storage capacities, and polyolefin fibers provide high tensile strength, abrasion, and solvent resistance. Preferred materials include blends of polyester and viscose fibers/wood pulp. In embodiments, the base material for the glove may not include cloth, plastic, or rubber, i.e., non-absorbent materials. The base material being the primary material that the glove is made from, not including, for example, accessory or ornamental features.

A disinfectant generally used in hand sanitizers can be used. The antimicrobial activity of disinfectants can occur in various ways including growth inhibition (e.g., bacteriostatic, fungistatic) and lethal action, i.e., sporicidal, bactericidal, fungicidal, and virucidal effects or properties. Disinfectants comprise a wide variety of active chemical agents including biocides. The active ingredients will generally include alcohols, chlorine, aldehydes, proxygenes, and quaternary ammonium compounds. The disinfectant composition may be virucidal for the SARS2-COV2 virus and its variants.

Alcohol is inexpensive and readily available and allows an efficient wetting of surfaces with a rapid bactericidal effect without toxicity issues. Hydrogen peroxide provides germicidal activity including bacterial spores with longer contact time. It is environmentally friendly. Peracetic acid (PAA) has a rapid action against all microorganisms including spores at low concentration. It is very efficient even in the presence of organic matter without producing residues.

Quaternary Ammonium Compounds (QACs) are commonly used disinfectants with good cleaning deodorization properties and a broad spectrum of biocidal and sporostatic activity including against lipids and enveloped viruses. Incorporation of quaternary ammonium compounds into polymers is effective against biofilm. Preferred disinfectants are alcohol, hydrogen peroxide, and hypochlorite.

Chlorine compound disinfectants include hypochlorite, chlorine dioxide and chloramine-T trihydrate. Hypochlorite is preferred especially because of its low cost and effectiveness. It displays a large bactericidal spectrum with no toxic residues, and it is not affected by water hardness. The disclosed gloves may include a pre-wetted towelette type material containing disinfectants, antiseptics, surfactants etc. in a sealed package ready for use for up to 1 month or more after opening. Shelf life may be longer.

In embodiments, the disclosed gloves may include a single layer of an absorbent or semi absorbent substrate infused with an antimicrobial or antiviral composition. Preferably, the antimicrobial or antiviral composition would be a composition suitable as an antibacterial, sanitizing, hand composition.

In embodiments, the disinfectant is infused or impregnated into the base material of the gloves. This provides disinfectant simultaneously contacting the wearer's hands as well as any surface that the outside of the gloves may encounter.

In embodiments, the gloves may be multi-layer. In multi-layer embodiments each layer may include a towel-like, absorbent type material, as described above, with the feature that the impregnated disinfectant solution is present on the inside surface of the glove and is present on the outside surface of the glove as well and thus contacts any surface that the outer surface of the glove's encounters.

Assuming the wearer's hand is in contact with the inside surface of the glove, the disinfectant will be in simultaneous contact with the wearer's hand and the outer surface of the glove and thereby any surfaces the outside of the glove encounters. In embodiments, the gloves may include two layers. In embodiments, the gloves may include three layers with each layer comprising a disinfectant impregnated therein.

A glove as used herein is a garment covering the hand. Gloves usually have separate sheaths or openings for each finger and the thumb. A glove may be a fingerless glove. Fingerless gloves may have one small opening rather than individual openings for each finger. A glove may be a gauntlet. Gloves which cover the entire hand or first but do not have separate finger openings or sheaths for the fingers are usually called mittens. A glove may be a hybrid of glove and mitten that contains open-ended sheaths for the four fingers and an additional compartment encapsulating four fingers.

The glove compartment may be lifted off the fingers and folded back to allow the individual fingers ease of movement and access while the hand remains covered. The mitten cavity may be stitched onto the back of the fingerless glove only, allowing it to be flipped over (e.g., held back by Velcro or a button) to transform the garment from a mitten to a glove. These hybrids are called convertible mittens or glittens, a combination of a glove and mitten. An ambidextrous glove is a glove that can fit a wearer's right hand or left hand. See Glove, Wikipedia, The Free Encyclopedia, Date of last revision: 15 Feb. 2021, herein incorporated by reference.

A hand sanitizing composition as used herein may be a liquid, gel or foam generally used to kill viruses, bacteria, and/or microorganisms (on the hands). Hand sanitizing solutions generally comprise an alcohol, povidone-iodine, benzalkonium chloride and/or triclosan. Hand sanitizing solutions are safe in contact with human skin and effectively kill viruses, bacteria, and/or microorganisms. The disinfectant or hand sanitizing solutions may further comprise a variety of surfactants, preservatives, enzymes, perfumes, lotions, softeners, etc., as commonly known and used in the art. The disinfectant and/or hand sanitizing composition may be virucidal for the SARS2-COV2 virus and its variants. See Hand sanitizer, Wikipedia, The Free Encyclopedia, Date of last revision: 2 Mar. 2021, herein incorporated by reference.

### Example

One example of making a glove of the disclosure is as follows. An absorbent or semi absorbent dry substrate material including polyester, polypropylene, cotton, wood pulp, and/or rayon fibers of 50-100 gsm (analogous to a material used in commercial antibacterial wipes) is formed into a glove by using a mold and then using machining and/or stitching methods. The glove is then infused or impregnated with an antimicrobial quaternary ammonium salt composition at between about 1 to 10 times its weight. The glove is then folded with other gloves and stored in a resealable container for use.

While this disclosure includes specific examples, it will be apparent after an understanding of the disclosure of this application has been attained that various changes in form and details may be made in these examples without departing from the spirit and scope of the claims and their equivalents.

I claim:

1. An antibacterial, antiviral, glove comprising:
a woven or nonwoven absorbent or semi-absorbent substrate material infused with a disinfectant composition, wherein the glove comprises one or more layers and each layer comprises the woven or nonwoven absorbent or semi-absorbent substrate material infused with a disinfectant composition.

2. The antibacterial, antiviral, glove of claim 1, wherein the disinfectant composition is virucidal for the SARS2-COV2 virus and variants.

3. The antibacterial, antiviral, glove of claim 1, wherein the disinfectant composition comprises a hand sanitizing composition.

4. The antibacterial, antiviral, glove of claim 1, wherein the disinfectant composition comprises an alcohol, hydrogen peroxide, and/or a hypochlorite.

5. The antibacterial, antiviral, glove of claim 1, wherein the woven or nonwoven absorbent or semi-absorbent substrate material comprises a cellulosic fiber and/or a thermoplastic fiber.

6. The antibacterial, antiviral, glove of claim 5, wherein the cellulosic fiber comprises cotton, wood pulp, viscose, and/or lyocell.

7. The antibacterial, antiviral, glove of claim 5, wherein the thermoplastic fiber comprises polyethylene terephthalate, and/or polypropylene.

8. The antibacterial, antiviral, glove of claim 1, wherein the glove is ambidextrous.

9. The antibacterial, antiviral, glove of claim 1, wherein the glove is a mitten.

10. The antibacterial, antiviral, glove of claim 1, wherein the glove is a fingerless glove.

11. The antibacterial, antiviral, glove of claim 1, wherein the disinfectant composition comprises a surfactant, preservative, enzyme, and/or a perfume.

12. An antibacterial, antiviral, glove comprising:
a woven or nonwoven absorbent or semi-absorbent substrate material infused or impregnated with a hand sanitizing composition, wherein the glove comprises one layer of a woven or nonwoven absorbent or semi-absorbent substrate material infused with the hand sanitizing composition, and the glove is ambidextrous.

13. The antibacterial, antiviral, glove of claim 12, wherein the hand sanitizing composition comprises an alcohol.
